(19) United States
(12) Patent Application Publication
(10) Pub. No.: US 2022/0106568 A1
(43) Pub. Date: Apr. 7, 2022

(54) STEM CELL GENERATOR AND CONSTRUCTION METHOD THEREFOR

(71) Applicant: EAST CHINA UNIVERSITY OF SCIENCE AND TECHNOLOGY, Shanghai (CN)

(72) Inventors: Changsheng LIU, Shanghai (CN); Kai DAI, Shanghai (CN); Jing WANG, Shanghai (CN); Qinghao ZHANG, Shanghai (CN); Shunshu DENG, Shanghai (CN); Guilong LI, Shanghai (CN)

(21) Appl. No.: 17/427,737

(22) PCT Filed: Jan. 21, 2020
(86) PCT No.: PCT/CN2020/073591

§ 371 Date: Aug. 2, 2021

(30) Foreign Application Priority Data
Jan. 31, 2019 (CN) 201910099941.8

(51) Int. Cl. C12N 5/0775; A61P 37/06; A61K 35/12

(52) U.S. Cl. CPC C12N 5/0662; A61K 35/12; A61P 37/06

(57) ABSTRACT

Disclosed are a stem cell generator, a construction method therefor and the use thereof. The stem cell generator is formed by implanting a biomaterial loaded with active substances and/or cells, or a biomaterial with an osteogenic induction capability into animal or human bodies and producing organoids upon development.

### TECHNICAL FIELD

The invention belongs to the cross field of materials, life and medicine, and relates to a stem cell generator and a construction method thereof.

### BACKGROUND

The human skeletal system not only provides the mechanical support, but also provides a suitable microenvironment for various pluripotent stem cells such as hematopoietic stem/progenitor cells and mesenchymal stem cells in the bone marrow tissue contained therein thereby ensuring the normal functions of stem cells, such as hematopoiesis development, bone regeneration, etc. There are two typical stem cells in bone marrow tissue, namely hematopoietic stem cells and mesenchymal stem cells.

Hematopoietic stem cells are a type of pluripotent stem cells with self-renewal and multi-lineage differentiation capabilities and are the most widely used type of stem cells in clinical applications so far. Hematopoietic stem cell transplantation (HSCT) therapy is a treatment method for patients with hematopoietic system damage, such as leukemia patients and patients with hematopoietic disorders after receiving chemotherapy and radiotherapy, to infuse healthy hematopoietic stem cells (HSC) to rebuild the patient's hematopoietic and immune system. Many clinical treatment results show that hematopoietic stem cell transplantation has a good effect on the treatment of various malignant hematological diseases, tumors, hematopoietic failure, severe radiation sickness, genetic diseases and other diseases.

Mesenchymal stem cells are a type of fibroblast-like pluripotent stem cells that can grow adherently. They are cultured in vitro and exhibit the ability to differentiate into osteoblasts, cartilage and adipogenesis. Because they are easy to separate and cultivate, and have strong plasticity, and have a wide range of sources, they can be used to treat diseases such as progeria, spinal cord injury, insomnia, ovarian injury, Alzheimer's disease, chronic wounds, liver cirrhosis, autoimmune diseases, etc. They are a type of stem cells most commonly used in the current stem cell therapy.

Existing methods for obtaining stem cells are usually in vitro methods, such as in vitro transformation and induction of embryonic stem cells, cord blood stem cells, adult stem cells to induced pluripotent stem cells (IPS), and in vitro culture and expansion of stem cells, etc., and the amount of acquisition is small. Bone marrow is the habitat of many kinds of stem cells. However, due to the various types of cells, multiple growth factors/cytokines and complex microenvironments involved in bone marrow, it cannot be imitated by in vitro methods.

### SUMMARY

The object of the present invention is to provide a stem cell generator produced by implanting a biomaterial loaded with active substance or a biomaterial with activity into the body to produce functionalized bone-like organs through developmental process, which contains various pluripotent stem cells including hematopoietic stem/progenitor cells and mesenchymal stem cells.

The first aspect of the present invention provides a stem cell generator, which is formed by implanting a biomaterial loaded with an active substance and/or cell, or a biomaterial with osteoinductive ability into an animal or human body to develop and generate an organoid.

In another preferred embodiment, the active substance is bone morphogenetic protein-2 (BMP-2), bone morphogenetic protein-7 (BMP-7), osteogenic peptide, growth factor or polypeptide having the ability to induce bone regeneration and angiogenesis, such as VEGF, PDG, or a combination of the growth factor/polypeptide.

In another preferred example, the bone morphogenetic protein-2 is recombinant bone morphogenetic protein-2.

In another preferred example, the bone morphogenetic protein-7 is recombinant bone morphogenetic protein-7.

In another preferred example, the cell is mesenchymal stem cell, and the mesenchymal stem cell is bone marrow-derived mesenchymal stem cell, adipose-derived mesenchymal stem cell, or mesenchymal stem cell from other sources; other type of cell having osteogenic differentiation ability; a cell assisting mesenchymal stem cell in osteogenic differentiation, such as vascular endothelial cell and the like.

In another preferred example, the biomaterial is selected from one of collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix, or a copolymer/blend composition thereof.

In another preferred example, the biomaterial is autologous bone or allogeneic bone.

In another preferred example, the animal or human body refers to the muscle pocket, muscle space, intra-muscle, subcutis, or dorsal muscle of the abdominal cavity of the animal or human.

In another preferred example, the stem cell generator contains T cells (CD3⁺), B cells (B220⁺), myeloid cells (CD11b⁺), red blood cells (Ter119⁺), hematopoietic progenitor cells (LKS−), and hematopoietic stem cells (LKS+).

In the present invention, the organoid has structures and functions similar to those of native bone, including complete bone tissue, bone marrow-like tissue and various functional stem cells.

In another preferred example, the organoid contains stem cell, and the stem cell is hematopoietic stem/progenitor cell, mesenchymal stem cell, endothelial progenitor cell or other types of pluripotent stem cell.

In the present invention, a biomaterial loaded with an active substance and/or with osteoinductive ability or a biomaterial with osteoinductive ability is implanted into an animal/human body, a special local microenvironment is created and an organoid with specific functions is formed after development for a certain period of time. The organoid has a function of producing stem cells, and functional stem cells can be produced in the organoid within a specific period of time.

Osteogenic activity growth factors represented by bone morphogenetic protein (BMP) have the effect of ectopic induction of osteogenesis. Under the combined action of the microenvironment in the body, they induce the development to produce specific organoid, which contains fully functional bone marrow, as well as a variety of pluripotent stem cells, thereby forming stem cell generator. The formed cells include complete hematopoietic precursor cells such as erythroid, myeloid and giant cells, and hematopoietic stem cells with long-term reconstruction ability, which can rebuild the hematopoietic system of mice irradiated with a lethal dose; moreover, the stem cell generator can also produce a large number of mesenchymal stem cells, the content of which is higher than that in normal bone marrow.

In another preferred example, the mass ratio of the active substance to the biomaterial is 0.0001-1:1.

In another preferred example, the number of cells inoculated is 1×10⁵-5×10⁸ cells per 100-150 mm³ of biomaterial.

The second aspect of the present invention provides a method for constructing the stem cell generator according to the first aspect, comprising the following steps:

(1) implanting a biomaterial into an animal or human body;

(2) generating an organoid after development in the body to form the stem cell generator, wherein,

the biomaterial is a biomaterial loaded with an active substance and/or cell, or a biomaterial having osteoinductive ability.

In another preferred example, the active substance is bone morphogenetic protein-2 (BMP-2), bone morphogenetic protein-7 (BMP-7), osteogenic peptide, other growth factor, polypeptide having ability to induce bone regeneration and angiogenesis, such as VEGF, PDG, or a combination of the growth factor/polypeptide.

In another preferred example, the bone morphogenetic protein-2 is recombinant bone morphogenetic protein-2.

In another preferred example, the bone morphogenetic protein-7 is recombinant bone morphogenetic protein-7.

In another preferred example, the cell is mesenchymal stem cell, and the mesenchymal stem cell is bone marrow-derived mesenchymal stem cell, adipose-derived mesenchymal stem cell, or mesenchymal stem cell from other sources; other type of cell having osteogenic differentiation ability; a cell assisting mesenchymal stem cell in osteogenic differentiation, such as vascular endothelial cell and the like.

In another preferred example, the biomaterial is selected from one of collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix, or a copolymer/blend composition thereof.

In another preferred example, the biomaterial is autologous bone or allogeneic bone.

In another preferred example, the animal or human body refers to the muscle pocket, muscle space, intra-muscle, subcutis, or dorsal muscle of the abdominal cavity of the animal or human.

In the present invention, the organoid has structures and functions similar to those of native bone, including complete bone tissue, bone marrow-like tissue and various functional stem cells.

In another preferred example, the organoid contains stem cell, and the stem cell is hematopoietic stem/progenitor cell, mesenchymal stem cell, endothelial progenitor cell or other type of pluripotent stem cell.

The third aspect of the present invention provides a method for enriching stem cells, comprising the following steps:

(1) implanting a biomaterial into an animal or human body;

(2) generating an organoid after development in the body and enriching stem cells, wherein

the biomaterial is a biomaterial loaded with an active substance and/or cell, or a biomaterial having osteoinductive ability.

In another preferred example, the active substance is bone morphogenetic protein-2 (BMP-2), bone morphogenetic protein-7 (BMP-7), osteogenic peptide, other growth factor or polypeptide having ability to induce bone regeneration and angiogenesis, such as VEGF, PDG, or a combination of the growth factor/polypeptide.

In another preferred example, the bone morphogenetic protein-2 is recombinant bone morphogenetic protein-2.

In another preferred example, the bone morphogenetic protein-7 is recombinant bone morphogenetic protein-7.

In another preferred example, the cell is mesenchymal stem cell, and the mesenchymal stem cell is bone marrow-derived mesenchymal stem cell, adipose-derived mesenchymal stem cell, or mesenchymal stem cell from other sources; other type of cell having osteogenic differentiation ability; a cell assisting mesenchymal stem cell in osteogenic differentiation, such as vascular endothelial cell and the like.

In another preferred example, the biomaterial is selected from one of collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix, or a copolymer/blend composition thereof.

In another preferred example, the biomaterial is autologous bone or allogeneic bone.

In another preferred example, the animal or human body refers to the muscle pocket, muscle space, intra-muscle, subcutis, or dorsal muscle of the abdominal cavity of the animal or human.

In the present invention, the organoid has structures and functions similar to those of native bone, including complete bone tissue, bone marrow-like tissue and various functional stem cells.

In another preferred example, the organoid contains stem cell, and the stem cell is hematopoietic stem/progenitor cell, mesenchymal stem cell, endothelial progenitor cell or other type of pluripotent stem cell.

The fourth aspect of the present invention provides use of the stem cell generator according to the first aspect in the manufacture of a material for the prevention and/or treatment of graft-versus-host disease or hematopoietic injury, or a material for bone marrow transplantation.

In the present invention, the stem cell is used in the manufacture of a medicament for treating hematopoietic injury.

In another preferred example, the hematopoietic injury is hematopoietic injury caused by radiotherapy or chemotherapy.

In another preferred example, the treatment is the transplantation of bone marrow cells produced in the stem cell generator. In another preferred example, the bone marrow cells are a single cell suspension made from cells in the stem cell generator.

In another preferred example, the bone marrow cells are derived from the organoid (stem cell generator) formed by implanting a biomaterial loaded with a growth factor and/or cell, or a biomaterial with osteoinductive ability into muscle pockets or subcutaneous parts of an animal or human and developing over a period of time.

In another preferred example, the cell used is an adipose-derived mesenchymal stem cell, bone marrow-derived mesenchymal stem cell, or other cell with osteogenic differentiation ability, or a combination thereof.

In another preferred example, the cells produced are hematopoietic stem/progenitor cells (HSC/HPC), mesenchymal stem cells (MSC) or other types of pluripotent stem cells.

In the present invention, the stem cell is also used in the manufacture of a medicament for promoting the recovery of blood cells and hematopoietic progenitor/stem cells after bone marrow failure caused by radiotherapy/chemotherapy.

In the present invention, the stem cell is also used in the manufacture of bone marrow transplantation material, a medicament for treating hematopoietic hypofunction, a medicament for treating leukopenia, or a medicament for treating acute or chronic leukemia.

In another preferred example, the stem cell generator can be used for the following occasions or disease treatment:

(1) bone marrow transplantation;

(2) promoting the recovery of hematopoietic system after radiotherapy/chemotherapy;

(3) treating blood system abnormalities, such as leukemia.

In another preferred example, the bone marrow cell is used before, during, or after radiotherapy or chemotherapy.

In another preferred example, the hematopoietic hypofunction is one caused by radiation or chemotherapy injury drugs or by bone marrow suppression.

In another preferred example, the manufacture comprises grinding the stem cell generator in a buffer, crushing, and passing through a cell sieve to obtain a single cell suspension.

The method for producing stem cells of the present invention is completely different from the existing methods for obtaining stem cells, for example transforming and inducing embryonic stem cells, umbilical cord blood stem cells, adult stem cells in vitro to form induced pluripotent stem cells (IPS), and culturing and expanding stem cells in vitro. The stem cell generator of the present invention is formed by inducing a biomaterial loaded with active substance or a biomaterial with activity in vivo to form a functionalized bone-like organ, which contains a variety of pluripotent stem cells including hematopoietic stem/progenitor cells, mesenchymal stem cells, etc.

The research results of the present invention show that the stem cell generator can induce or highly enrich pluripotent stem cells such as hematopoietic progenitor/stem cells and mesenchymal stem cells, and the induced or highly enriched pluripotent stem cells have complete functions and can be used for scientific research or clinical treatment that requires related stem cells. The method of the present invention is a brand-new method for producing/obtaining stem cells, opens up a brand-new way for obtaining stem cells, and has important scientific significance and broad application prospects.

It should be understood that within the scope of the present invention, the above-mentioned each technical feature of the present invention and each technical feature specifically described thereafter (such as the examples) can be combined with each other to form a new or preferred technical solution. Each feature disclosed in the specification can be replaced by any alternative feature that provides the same, equal or similar purpose. Due to space limitations, they will not be repeated one by one.

### BRIEF DESCRIPTION OF THE FIGURES

FIG. 1 shows the organoids induced by different doses of rhBMP-2 at 1 week and 3 weeks, which are stem cell generators.

FIG. 2 shows the H&E section of the stem cell generator induced by 30 μg of rhBMP-2 at 1 week and 3 weeks.

FIG. 3 shows the stem cell generator produced by implanting subcutaneously the composite collagen sponge loaded with human bone marrow mesenchymal stem cells in NCG mice for 8 weeks.

FIG. 4 shows the H&E section of the stem cell generator produced by implanting subcutaneously the composite collagen sponge loaded with human bone marrow mesenchymal stem cells in NCG mice for 8 weeks.

FIG. 5 and FIG. 6 show the typical flow cytometric diagram and proportion chart of each line of blood cells in the stem cell generator induced by loading 30 μg of rhBMP-2 at 3 weeks, respectively.

FIG. 7 and FIG. 8 show the typical flow cytometric diagram and proportion chart of the hematopoietic progenitor/stem cells in the stem cell generator induced by loading 30 μg of rhBMP-2 at 3 weeks, respectively.

FIG. 9 and FIG. 10 show the typical flow cytometric diagram and proportion chart of the hematopoietic progenitor/stem cells in the stem cell generator induced by loading 10 μg of rhBMP-7 at 3 weeks, respectively.

FIG. 11 shows typical flow cytometry diagrams at different time points when the hematopoietic stem cells produced in the stem cell generator are used for long-term competitive reconstruction.

FIG. 12 shows the CD45.1 cell reconstitution ratio at different time points when hematopoietic stem cells produced in the stem cell generator are used for long-term competitive reconstitution.

FIG. 13, FIG. 14, and FIG. 15 show the B cell (B220+ cell), T cell (CD3+ cell), and myeloid cell (CD11b+ cell) reconstitution ratio at different time points when the hematopoietic stem cells produced in the stem cell generator are used for long-term competitive reconstruction, respectively.

FIG. 16 and FIG. 17 show the typical flow cytometric diagram and proportion chart of the mesenchymal stem cells in the stem cell generator induced by a biomaterial loaded with 30 μg of rhBMP-2 at 1 week and 3 weeks, respectively.

FIG. 18, FIG. 19, FIG. 20, and FIG. 21 show a macroscopic view, an H&E slice view, a typical flow cytometric diagram, and a flow statistic graph of the stem cell generator produced after material implantation, respectively.

FIG. 22, FIG. 23 and FIG. 24 show the changes in the weight of mice after tail vein injection of cells at the irradiation doses of 6.0 Gy, 7.0 Gy, and 8.0 Gy, respectively.

FIG. 25, FIG. 26 and FIG. 27 show the changes in the number of white blood cells in mice after tail vein injection of cells at the irradiation doses of 6.0 Gy, 7.0 Gy, and 8.0 Gy, respectively.

FIG. 28, FIG. 29 and FIG. 30 show the changes in the number of red blood cells in mice after tail vein injection of cells at the irradiation doses of 6.0 Gy, 7.0 Gy, and 8.0 Gy, respectively.

FIG. 31, FIG. 32 and FIG. 33 show the changes in the number of platelets in mice after tail vein injection of cells at the irradiation doses of 6.0 Gy, 7.0 Gy, and 8.0 Gy, respectively.

FIG. 34, FIG. 35, FIG. 36, and FIG. 37 show a macroscopic view, an H&E slice view, a typical flow cytometric diagram, and a flow statistic graph of the stem cell generator produced 8 weeks after the material of Example 10 was implanted, respectively.

### MODES FOR CARRYING OUT THE INVENTION

After extensive and intensive researches, the inventors of the present application found that an organoid can be formed to form stem cell generator after a biomaterial with osteoinductive ability (such as autologous bone, allogeneic bone, etc.) or a biomaterial loaded with active substance and/or cell (such as BMP-2, BMP-7 or mesenchymal stem cell) is implanted in the body and then subjected to development and the stem cell generator contains various lines of hematopoietic cells and hematopoietic progenitor/stem cells, as well as a high proportion of mesenchymal stem cells. These stem cells have complete functions and can be further used for scientific research and clinical applications of stem cells. Moreover, corresponding stem cell therapy can be developed based on various pluripotent stem cells such as hematopoietic stem/progenitor cells or mesenchymal stem cells in the stem cell generator. On this basis, the present invention has been completed.

### Term

“Stem cell generator” is an organoid with specific functions produced by implanting a biomaterial loaded with active substance and/or cell or a biomaterial with osteoinductive activity into an animal/human body and then subjecting to developmental process in a special local microenvironment for a certain period of time, in which various functionalized stem cells can be enriched.

In the present invention, the organoid has structures and functions similar to native bone, including complete bone tissue, bone marrow-like tissue and various functional stem cells.

Organoid is usually constructed using in vitro methods. Bone marrow is the habitat of various stem cells. However, due to the various types of cells, multiple growth factors/cytokines and complex microenvironments involved in bone marrow, in vitro methods cannot be imitated. The present invention adopts the construction method in vivo, uses material and active molecule to form an organoid, studies the components therein, and proposes a method for enriching stem cells of the present invention.

The present invention will be further described below in conjunction with specific examples. It should be understood that these examples are only used to illustrate the present invention and not to limit the scope of the present invention. The experimental methods without specific conditions in the following examples generally follow the conventional conditions (such as those described in Sambrook et al., Molecular Cloning: Laboratory Manual (New York: Cold Spring Harbor Laboratory Press, 1989) or the conditions recommended by the manufacturer. Unless stated otherwise, percentages and parts are percentages by weight and parts by weight.

Unless otherwise defined, all professional and scientific terms used herein have the same meaning as those familiar to the skilled in the art. In addition, any methods and materials similar to or equivalent to those described can be applied to the method of the present invention. The preferred implementation methods and materials described herein are for demonstration purposes only.

### Example 1 Preparation of Implant Material

Recombinant human bone morphogenetic protein-2 (rhBMP-2) was synthesized by using eukaryotic or prokaryotic expression system (Optimized DNA sequences encoding recombinant human bone morphogenetic protein-2 (rhBMP-2), preparation method and the uses there of. U.S. Pat. No. 7,947,821 B2; Liu Changsheng et al., ZL 200610118006.4; ZL200910045832.4).

Recombinant human bone morphogenetic protein-2 was added at different doses (10 μg, 30 μg, 80 μg, 200 μg) to 5×5×5 mm gelatin sponge (10 mg), and lyophilized to form an active material containing growth factor.

10 μg of recombinant human bone morphogenetic protein-7 (rhBMP-7) synthesized by eukaryotic or prokaryotic expression system was added to 5×5×5 mm gelatin sponge (10 mg), and lyophilized to form an active material containing growth factor.

A concentrated solution containing 1×10⁶ of the third-generation human mesenchymal stem cells (hMSCs) was inoculated to a 5×5×5 mm collagen sponge material (10 mg), placed in a 37′C incubator and incubated for 2 h to form a cell-containing active material.

### Example 2

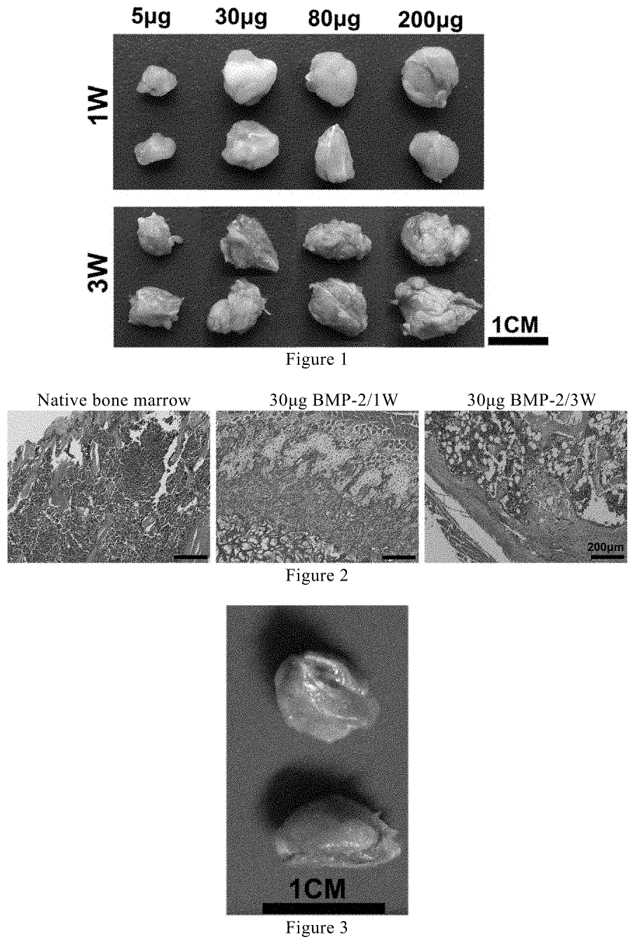

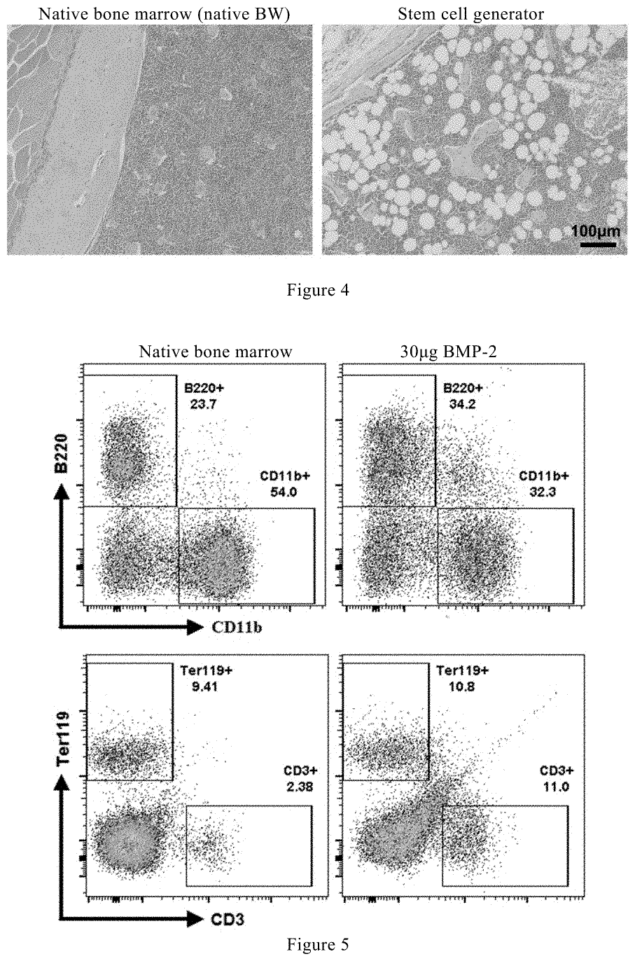

The active material containing rhBMP-2 of Example 1 was implanted into the body to develop a stem cell generator. Different doses of rhBMP-2 (10 μg, 30 μg, 80 μg, 200 μg) were loaded into gelatin sponge material, and implanted into the thigh muscle space of C57BL/6 male mice. After 3 weeks of feeding, the formed organoids, i.e., stem cell generators, were taken out. One part was used for macro-photographs and histological evaluation (FIG. 1 and FIG. 2). After the muscles attached to the surface were removed, the other part was placed in a mortar containing a little PBS buffer, and the organoid (stem cell generator) was crushed with a pestle and then passed through a cell sieve to obtain a single cell suspension. The resulting single cell suspension could be used in subsequent experiments.

FIG. 1 showed the stem cell generators produced by implanting different doses of rhBMP-2 at 1 week and 3 weeks. It could be seen from general observation that it was bone-like tissue.

FIG. 2 showed the H&E section of the stem cell generator induced by 30 μg of rhBMP-2 at 1 week and 3 weeks. It could be seen that at 1 week, chondrocytes appeared in the organoid (stem cell generator), and at 3 weeks, obvious bone marrow-like tissue appeared in the organoid (stem cell generator).

### Example 3

The active material containing rhBMP-7 of Example 1 was used to generate a stem cell generator in vivo.

10 μg of rhBMP-7 was loaded in gelatin sponge material and implanted into the thigh muscle of C57BL/6 male mice. After 3 weeks of feeding, the stem cell generator was taken out. The muscles attached to the surface were removed, and then the stem cell generator was placed in a mortar containing a little Hank's balanced salt solution (HBSS), crushed with a pestle, and passed through a cell sieve to obtain a single cell suspension. The resulting single cell suspension could be used in subsequent experiments.

### Example 4

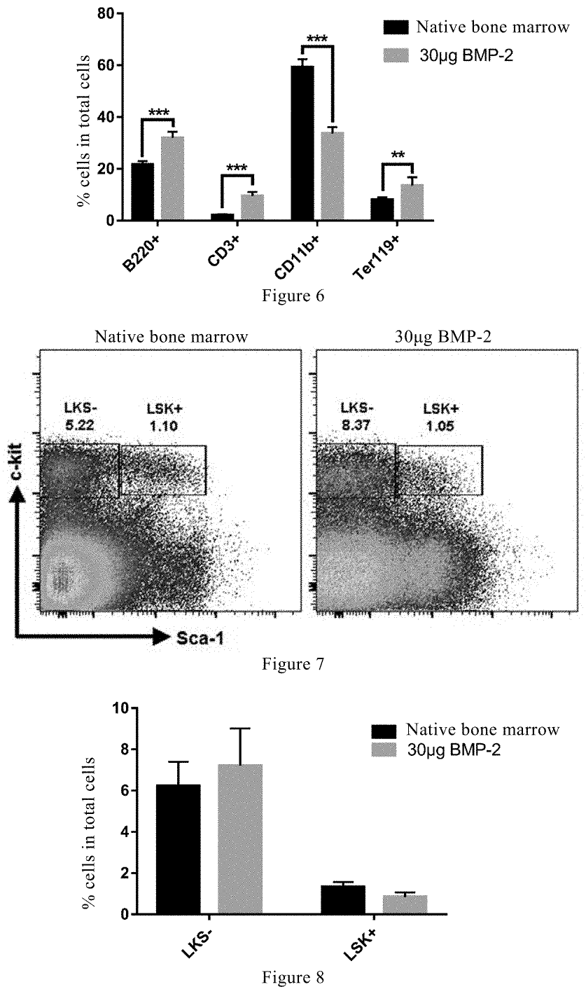

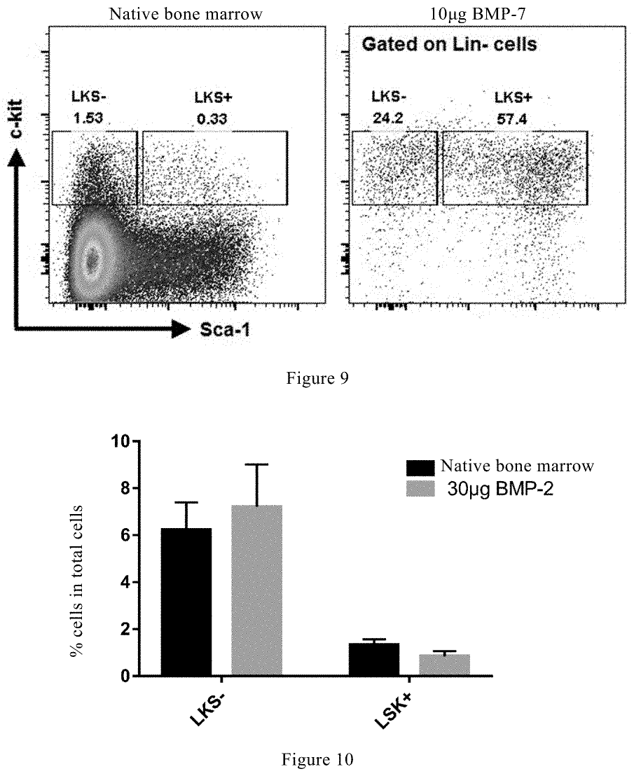

The collagen sponge loaded with human bone marrow mesenchymal stem cells of Example 1 was implanted into the thigh muscle of male NCG immunodeficient mice. After 3 weeks of feeding, the stem cell generator was taken out. One part was used for macro-photographs and histological evaluation (FIG. 3 and FIG. 4). After the muscles attached to the surface were removed, the other part was placed in a mortar containing a little buffer, and the stem cell generator was crushed with a pestle and passed through a cell sieve to obtain a single cell suspension. The resulting single cell suspension could be used in subsequent experiments.

### Example 5 Detection of the Content of the Cells of Each Blood Line and Hematopoietic Progenitor/Stem Cells Contained in the Stem Cell Generator Induced by rhBMP-2

The purpose of this example is to detect the proportion of the cells of each blood line and hematopoietic progenitor/stem cells in the stem cell generator, and to compare the content with the corresponding cells of normal bone marrow, to prove that the stem cell generator has a fully functional hematopoietic system containing cells of each blood line and hematopoietic progenitor/stem cells and can provide treatment for abnormal hematopoietic function.

The active material containing rhBMP-2 was the gelatin sponge scaffold loaded with 30 μg of rhBMP-2 prepared in Example 1.

The single cell suspension was a single cell suspension prepared according to the method of Example 2.

Method: C57BL/6 mice (SPF grade, male, 8 weeks old) were randomly grouped, and then the material containing 30 μg of rhBMP-2 prepared in Example 1 was implanted into the thigh muscle. After 3 weeks of feeding, the formed organoid (i.e., stem cell generator) was taken out. After the muscles attached to the surface were removed, the obtained stem cell generator was placed in a mortar containing a little PBS buffer and the organoid (stem cell generator) was crushed with a pestle and passed through a cell sieve to obtain a single cell suspension. The resulting single cell suspension could be used in subsequent flow cytometry experiments. The experiment groups (list) were as follows.

| | Native bone | stem cell |
|---|---|---|
| Group | marrow group | generator group |
| Number | 5 | 5 |

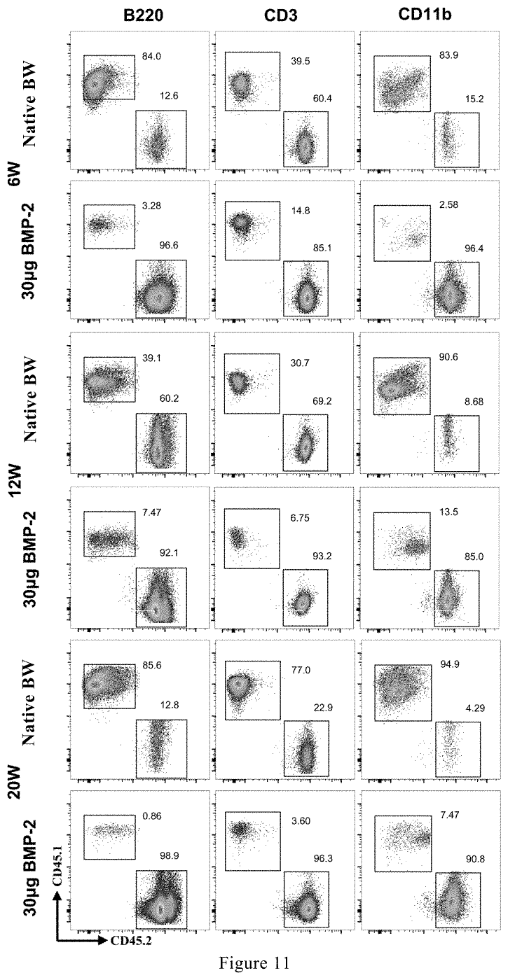

FIG. 5 and FIG. 6 showed the typical flow cytometric diagram and the corresponding proportion chart of cells of each blood line in the stem cell generator induced by rhBMP-2.

The typical flow cytometric diagram of FIG. 5 showed that there were complete blood line cells in the stem cell generator, which contained T cells (CD3⁺), B cells (B220⁺), myeloid cells (CD11b⁺), and red blood cells (Ter119⁺).

FIG. 6 showed that the proportions of B cells, red blood cells, and T cells in the stem cell generator were significantly higher than that in the native bone marrow group, while the proportion of myeloid cells was significantly lower than that in the native bone marrow group, indicating that the stem cell generator had complete blood cell line, but the proportion was not completely consistent with that of the native bone marrow group.

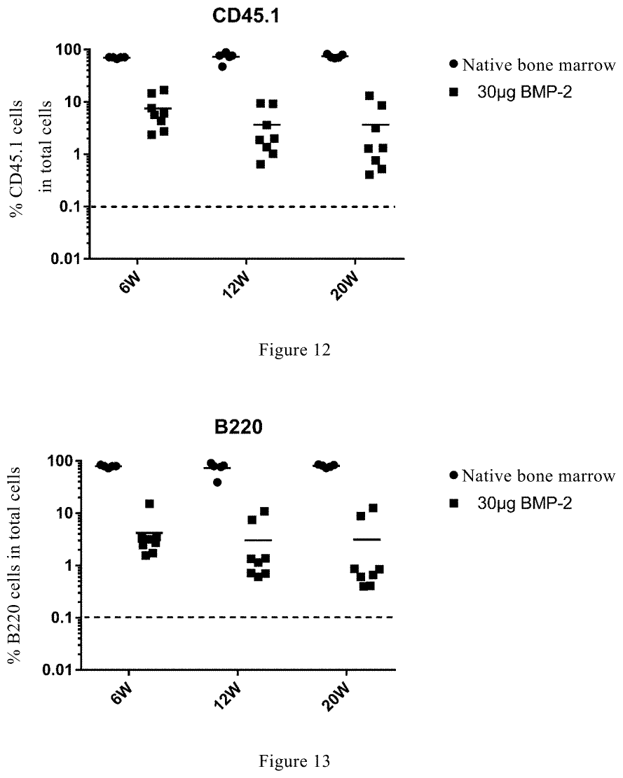

FIG. 7 and FIG. 8 showed the typical flow cytometric diagram and proportion chart of the hematopoietic progenitor/stem cells in the stem cell generator induced by rhBMP-2. The typical flow cytometric diagram in FIG. 7 showed that there were complete hematopoietic progenitor/stem cells in the stem cell generator, which contained hematopoietic progenitor cells (LKS−) and hematopoietic stem cells (LKS+). FIG. 8 showed that there was no significant difference between hematopoietic progenitor/stem cells in the stem cell generator and those in the native bone marrow group. It showed that the stem cell generator had complete hematopoietic progenitor/stem cells, which could provide cells for the treatment of bone marrow injury and hematopoietic hypofunction after radiotherapy and chemotherapy.

### Example 6 Detection of the Content of the Hematopoietic Progenitor/Stem Cells Contained in the Stem Cell Generator Induced by Active Material Containing 10 μg of rhBMP-2

The purpose of this example is to detect the proportion of the hematopoietic progenitor/stem cells in the stem cell generator, and to compare the content with the corresponding cells of normal bone marrow, to prove that the bone marrow in the stem cell generator has a fully functional hematopoietic system, contains hematopoietic progenitor/stem cells and can be used in the treatment for abnormal hematopoietic function.

The active material containing rhBMP-7 was the gelatin sponge scaffold loaded with 10 μg of rhBMP-7 prepared in Example 1.

The single cell suspension was a single cell suspension prepared according to the method of Example 3.

Method: C57BL/6 mice (SPF grade, male, 8 weeks old) were randomly grouped, and then the prepared material containing 10 μg of rhBMP-7 was implanted into the thigh muscle. After 3 weeks of feeding, the formed organoid (i.e., stem cell generator) was taken out. After the muscles attached to the surface were removed, the obtained stem cell generator was placed in a mortar containing a little PBS buffer and the organoid (stem cell generator) was crushed with a pestle and passed through a cell sieve to obtain a single cell suspension. The resulting single cell suspension could be used in subsequent flow cytometry experiments. The experiment groups (list) were as follows.

| | Native bone | stem cell |
|---|---|---|
| Group | marrow group | generator group |
| Number | 5 | 5 |

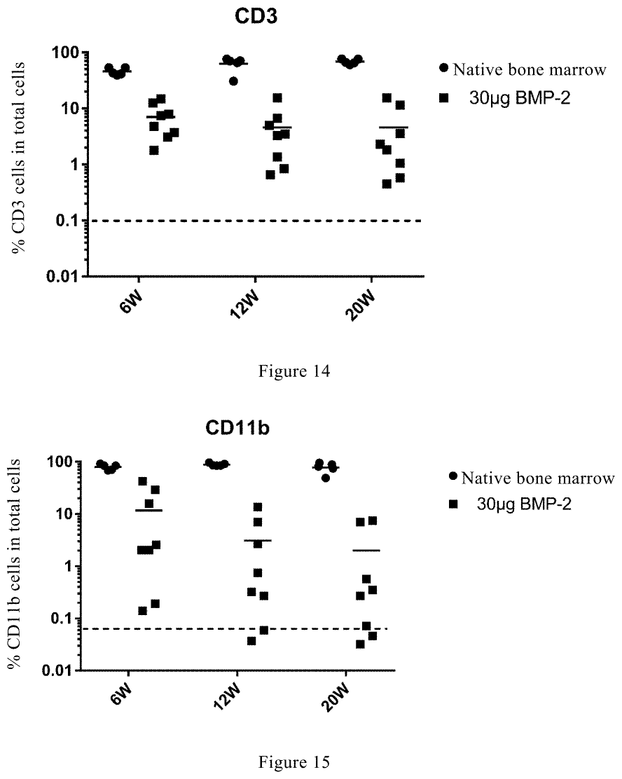

FIG. 9 and FIG. 10 showed the typical flow cytometric diagram and proportion chart of the hematopoietic progenitor/stem cells in the stem cell generator induced by rhBMP-7. The typical flow cytometric diagram in FIG. 9 showed that there were complete hematopoietic progenitor/stem cells in the stem cell generator, which contained hematopoietic progenitor cells (LKS−) and hematopoietic stem cells (LKS+). FIG. 10 showed that the contents of hematopoietic progenitor/stem cells in the stem cell generator were significantly higher than that in the native bone marrow group, indicating that the stem cell generator had abundant hematopoietic progenitor/stem cells and could provide cells for the treatment of abnormal hematopoietic function.

### Example 7 Evaluation of Pluripotency of Hematopoietic Stem/Progenitor Cells in Stem Cell Generator Induced by Active Material Containing rhBMP-2

The purpose of this example is to evaluate the pluripotency of hematopoietic stem/progenitor cells in the stem cell generator induced by rhBMP-2, by blending the cells (CD45.1) in the stem cell generator with the native bone marrow cells (CD45.2) and then reconstructing competitively the hematopoietic system of mice (CD45.2) destroyed by 10 Gy X-ray irradiation over a long period of time. If the cells from the stem cell generator account for more than 0.1% of the blood cells, it is regarded that the hematopoietic stem/progenitor cells in the stem cell generator are pluripotent and a realistic basis for the treatment of abnormal hematopoietic function is provided.

The active material containing rhBMP-2 was the gelatin sponge scaffold loaded with 30 μg of rhBMP-2 prepared in Example 1.

Method: C57BL/6 CD45.1 male mice (SPF grade, 8 weeks old) were randomly grouped, and then the material containing 30 μg of rhBMP-2 prepared in Example 1 was implanted into the thigh muscle. After 3 weeks of feeding, the formed organoid (i.e., stem cell generator) was taken out. After the muscles attached to the surface were removed from the obtained stem cell generator and femur or iliac bone, the stem cell generator (organoid) and femur or iliac bone were placed in a mortar containing a little PBS buffer and crushed with a pestle, passed through a cell sieve to obtain a single cell CD45.1 suspension of stem cell generator and a single cell CD45.1 suspension of native bone marrow, respectively. In addition, femur and ilium bone marrow were taken from SPF C57BL/6 CD45.2 mice, 8 weeks old, to prepare a single cell CD45.2 suspension. 1×10⁶ CD45.1 cells from stem cell generator or native bone marrow and 2×10⁵ CD45.2 cells were mixed to make two sets of 200 μL single cell suspensions, respectively and then transplanted into CD45.2 receptor mice irradiated with 10 Gy X-rays. The obtained single cell suspension was used for subsequent stem cell transplantation experiments. Then, at 6, 12 and 20 weeks, the peripheral bloods of each group of receptor mice were collected for flow cytometry to evaluate the pluripotency of the hematopoietic stem cells contained in the stem cell generator. The experiment groups (list) were as follows.

| | Native bone | stem cell |
|---|---|---|
| Group | marrow group | generator group |
| Number | 5 | 8 |

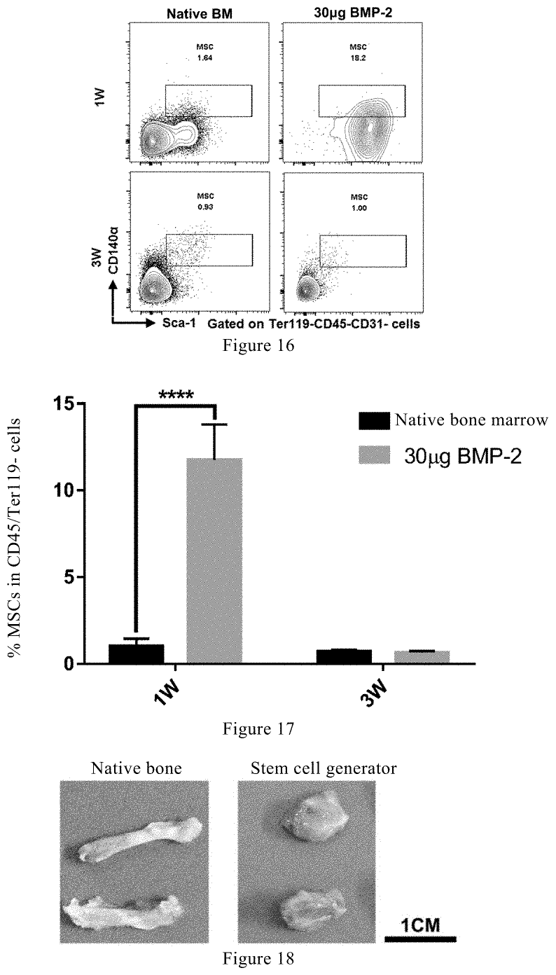

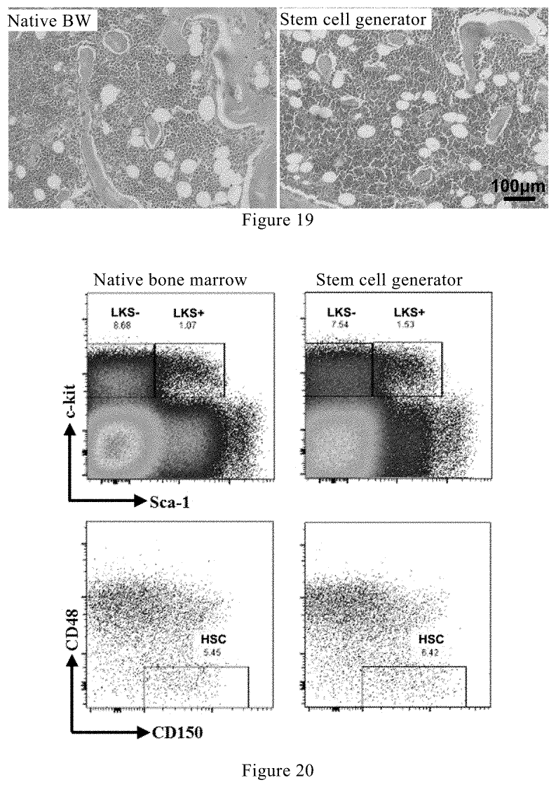

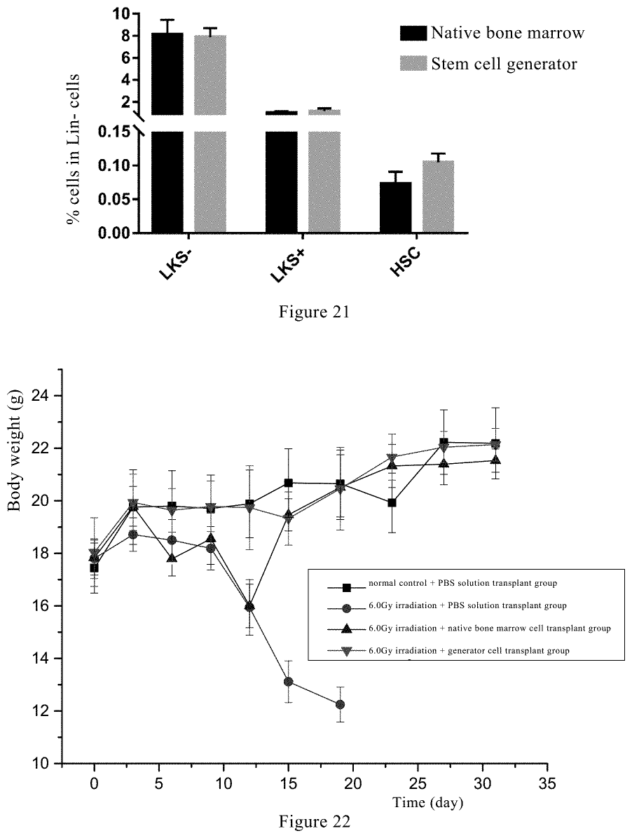

FIGS. 11-15 showed the typical flow cytometric diagram and the corresponding proportion chart of cells of each blood line in the stem cell generator induced by rhBMP-2 at different time points in the long-term competitive hematopoietic reconstitution experiment. FIG. 12 was a graph showing changes in the proportion of CD45.1 cells at different time points. It could be seen that the proportion of stem cell generator-derived cells at all time points was greater than 0.1%, namely, the hematopoietic stem/progenitor cells derived from the stem cell generator had long-term hematopoietic reconstitution ability. FIGS. 13-14 had the same trend as FIG. 12, and only part of the stem cell generator-derived myeloid cells of FIG. 15 had a cell reconstitution ratio of less than 0.1% at 12 and 20 weeks. In general, the hematopoietic stem/progenitor cells in the stem cell generator had long-term hematopoietic reconstruction ability, namely, were pluripotent stem cells.

### Example 8 Detection of the Content of Bone Marrow Mesenchymal Stem Cells in the Stem Cell Generator Induced by rhBMP-2

The purpose of this example is to detect the proportion of bone marrow mesenchymal stem cells in the stem cell generator induced by biomaterial containing rhBMP-2, so as to use mesenchymal stem cells to treat bone defect repair, cartilage defect repair, graft-versus-host disease (GVHD) and other diseases.

The active material containing rhBMP-2 was the gelatin sponge scaffold containing 30 μg of rhBMP-2 prepared in Example 1.

The single cell suspension was a single cell suspension prepared from the stem cell generator induced in the scaffold containing 30 μg of rhBMP-2 in Example 2.

Method: C57BL/6 mice (SPF grade, male, 8 weeks old) were randomly grouped, and then the material containing 30 μg of rhBMP-2 prepared in Example 1 was implanted into the thigh muscle. After 3 weeks of feeding, the formed organoid (i.e., stem cell generator) was taken out. After the muscles attached to the surface were removed, the obtained stem cell generator was placed in a mortar containing a little PBS buffer and the organoid (stem cell generator) was crushed with a pestle and passed through a cell sieve to obtain a single cell suspension. The resulting single cell suspension could be used in subsequent flow cytometry experiments. The experiment groups (list) were as follows.

| | Native bone | stem cell |
|---|---|---|
| Group | marrow group | generator group |
| Number | 5 | 5 |

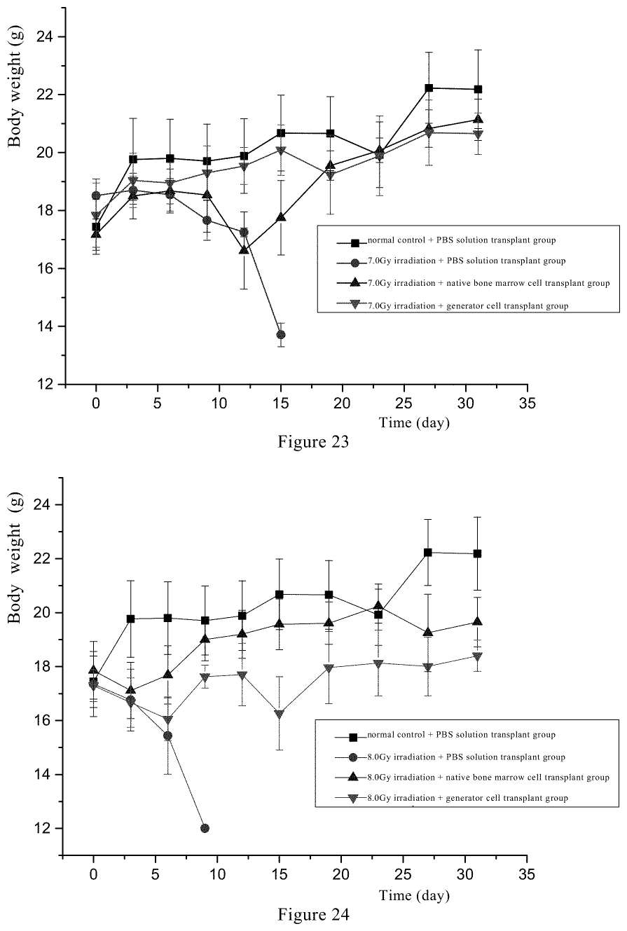

FIG. 16 and FIG. 17 showed the typical flow cytometric diagram and proportion chart of the bone marrow mesenchymal stem cells in the stem cell generator induced by active material containing rhBMP-2. It could be seen from FIG. 17 that at 1 week, the content of mesenchymal stem cells in the stem cell generator was significantly higher than that in the native bone marrow, and at 3 weeks, the content of mesenchymal cells in the stem cell generator approached that in the native bone marrow. It could be seen that a large number of mesenchymal stem cells were enriched in the stem cell generator, and enriched mesenchymal stem cells had great potential value for the treatment of bone defect repair, cartilage defect repair, graft versus host disease (GVHD) and other diseases.

### Example 9 Investigation on the Effect of Bone Marrow Cells in the Stem Cell Generator on Promoting Hematopoietic Recovery in Radiation-Damaged Mice

Method: 30 μg of recombinant human bone morphogenetic protein-2 (rhBMP-2) synthesized by eukaryotic or prokaryotic expression system was added to gelatin sponge (10 mg), and then lyophilized to form an active material containing growth factor. The prepared material was implanted into the thigh muscle pocket of an 8-week-old C57BL/6 male mouse. After 3 weeks of feeding, the stem cell generator and the native bone were taken out. After the muscles attached to the surface were removed from a part of the stem cell generator or native bone, a part of the stem cell generator and native bone were placed in a mortar containing a little PBS buffer, crushed with a pestle and passed through a cell sieve to obtain a single cell suspension, respectively. One part was prepared into 200 μL single cell suspension for bone marrow transplantation; the other part of stem cell generator and native bone were used to take macro photos and make H&E sections.

Bone marrow transplantation: C57BL/6 mice (SPF-grade, female, 8 weeks old) were randomly grouped. The single cell suspension prepared in the previous step was further transplanted into different groups of mice through the tail vein.

The experiment groups were as follows.

| Group | | Injected material | Number |
|---|---|---|---|
| normal control + PBS solution transplant group | | PBS solution | 10 |
| 6 Gy | irradiation + PBS solution transplant group | PBS solution | 5 |
| irradiation | irradiation + native bone marrow cell transplant | Native bone | 5 |
| | group | marrow suspension | |
| | irradiation + generator cell transplant group | generator bone | 5 |
| | | marrow suspension | |
| 7 Gy | irradiation + PBS solution transplant group | PBS solution | 5 |
| irradiation | irradiation + native bone marrow cell transplant | Native bone | 5 |
| | group | marrow suspension | |
| | irradiation + generator cell transplant group | generator bone | 5 |
| | | marrow suspension | |
| 8 Gy | irradiation + PBS solution transplant group | PBS solution | 5 |
| irradiation | irradiation + native bone marrow cell transplant | Native bone | 5 |
| | group | marrow suspension | |
| | irradiation + generator cell transplant group | generator bone | 5 |
| | | marrow suspension | |

Mouse radiotherapy injury model: The mice were subjected to one-time cobalt-60 irradiation according to the irradiation dose given in the grouping table, namely 0 Gy irradiation, 6 Gy irradiation, 7 Gy irradiation, and 8 Gy irradiation.

Intervention treatment: 24 hours after irradiation, the irradiated mice in the corresponding group were given intervention treatment, namely, by injecting 200 μL PBS solution, 200 μL native bone marrow cell suspension, 200 μL stem cell generator cell suspension through tail vein, wherein, native bone marrow cell suspension or stem cell generator cell suspension was the single cell suspension prepared by the method described in Example 4. Afterwards, the peripheral bloods of each group of mice were collected by sampling orbital bloods at the set sampling point for blood phase detection to observe the treatment effect. The blood test indicators were as follows.

(1) Detecting the number of white blood cells (WBC) in peripheral blood of each group continuously on the 3^{rd} day, the 6^{th} day, . . . (every 3 days, for 30 consecutive days);

(2) Detecting the number of red blood cells (RBC) in peripheral blood of each group continuously on the 3^{rd} day, the 6^{th} day, . . . (every 3 days, for 30 consecutive days);

(3) Detecting the number of platelets (PLT) in peripheral blood of each group continuously on the 3^{rd} day, the 6^{th} day, . . . (every 3 days, for 30 consecutive days);

(4) Detecting the weight of each group continuously on the 3^{rd} day, the 6^{th} day, . . . (every 3 days, for 30 consecutive days).

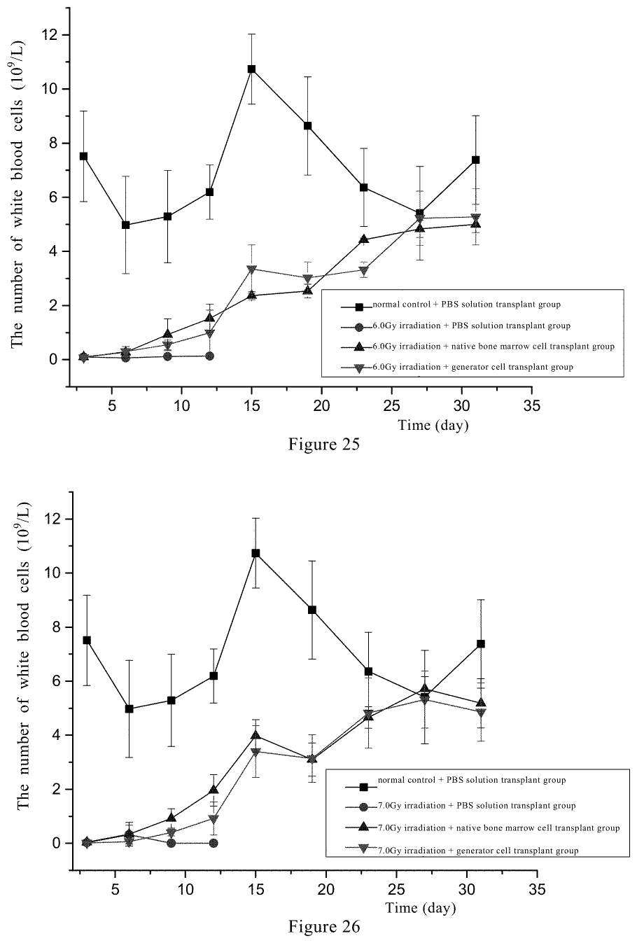

FIG. 18 showed a digital photo of the stem cell generator 8 weeks after implantation of the material into the muscle pocket. It could be seen that the color of the stem cell generator was similar to that of the native bone, which implied that it contained a large number of red blood cells and had a bone-like morphology, but the volume was bigger than native bone. H&E section of the stem cell generator in FIG. 19 further confirmed that the microstructure of the stem cell generator was similar to that of native bone, and the bone marrow cavity was filled with bone marrow cells and blood vessels.

FIG. 20 and FIG. 21 showed the flow cytometry correlation analysis of stem cell generator. It could be seen that the stem cell generator and the native bone had similar cell composition, and there was no significant difference between the proportion of LKS− cells, LSK+ cells and hematopoietic stem cells (HSCs) contained in the stem cell generator and the proportion of corresponding cells in the native bone marrow.

The examples illustrate that the constructed stem cell generator had a structure and function similar to native bone marrow, and the hematopoietic stem/progenitor cells contained therein have the potential to treat abnormal hematopoietic function.

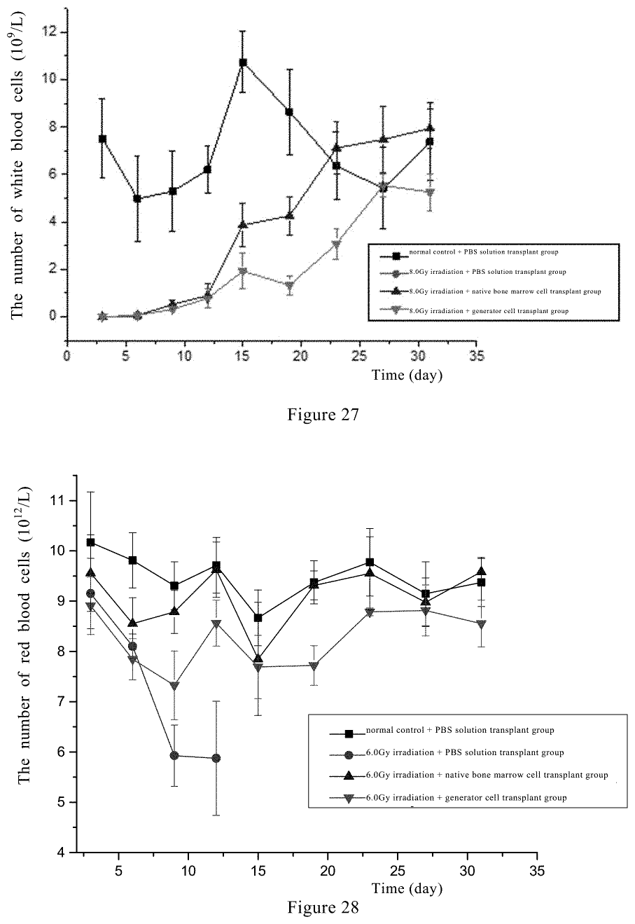

In order to further verify the therapeutic effect of the hematopoietic stem cells contained in the stem cell generator on the hematopoietic injury caused by radiotherapy, the mice were subjected to one-time cobalt-60 irradiation according to the irradiation dose given in the grouping table (0 Gy, 6 Gy, 7 Gy, 8 Gy). FIGS. 22-24 showed the changes in body weight of the mouse model at different irradiation doses after treatment.

The mice were injected with 200 μL of single cell suspension of bone marrow of the same species produced by the stem cell generator through the tail vein immediately after they were irradiated with cobalt 60 (6.0 Gy). FIG. 22 showed that the body weight of the irradiated control group did not change much from 0 to 9 days compared with the normal control group, but decreased sharply after 9 days until death. On the contrary, the weight change of the irradiation treatment group maintained roughly the same change trend as that of the normal control group.

The change trends of body weight (7.0 Gy and 8.0 Gy of cobalt 60 irradiation) in FIG. 23 and FIG. 24 were roughly the same as those shown in FIG. 26 and FIG. 27. It was particularly important to point out that due to the excessive radiation dose, the death rate of the irradiated control group had reached 100% within 9 days, and although the treatment group had steadily increased, it still had a gap with the normal control group.

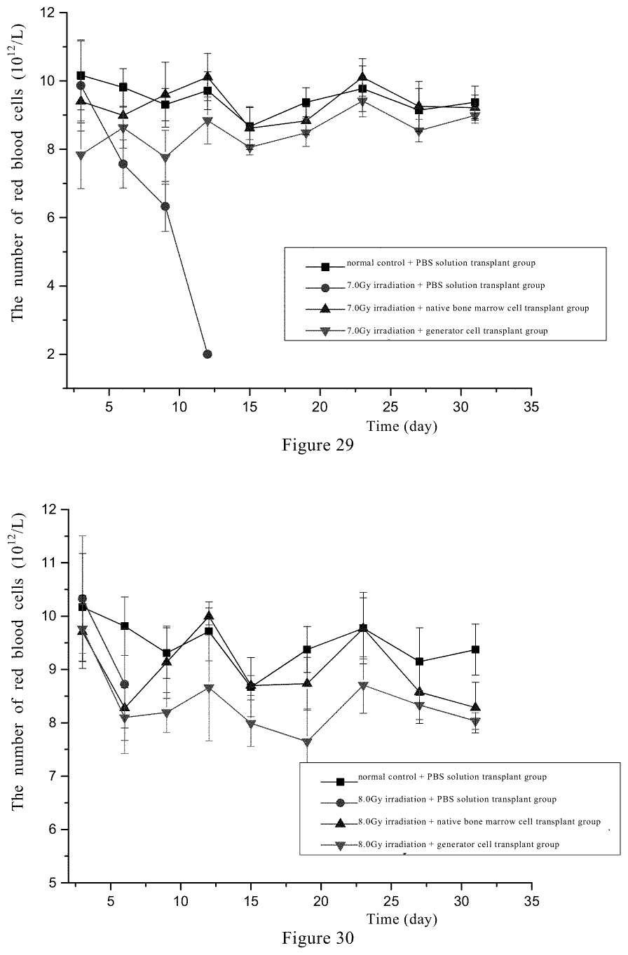

FIGS. 25-27 showed the changes in the number of white blood cells in injured mice received different doses of irradiation after treatment. It could be seen from FIG. 25 that the number of white blood cells in the irradiated control group and the treatment group after irradiation dropped sharply to 0, but the number of white blood cells in the treatment group increased steadily over time, and it was equal to the normal control group after 30 days, while the number in irradiated control group was still 0. It showed that after treatment, the hematopoietic function of irradiated mice was restored and the number of white blood cells increases steadily. The change trends of the number of white blood cells in FIGS. 26 and 27 (7.0 Gy and 8.0 Gy of cobalt 60 irradiation) were almost the same as those in FIGS. 23 and 24.

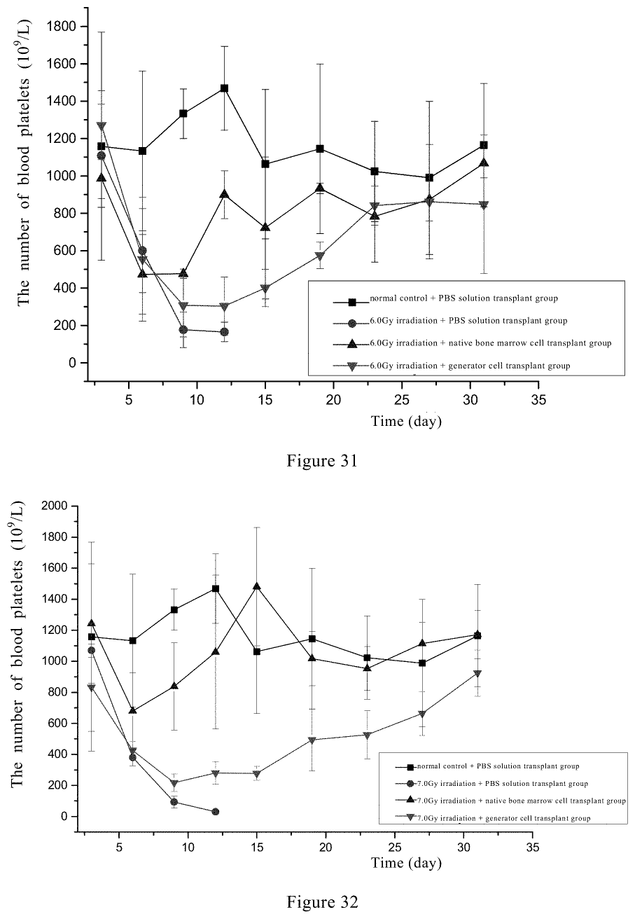

FIGS. 28-30 showed the changes in the number of red blood cells in the mouse model received different doses of irradiation after treatment. It could be seen from FIG. 28 that the numbers of red blood cells in the treatment group and the normal control group maintained the same change trend after the tail vein injection treatment after the irradiation, and there was no big numerical difference. The number of red blood cells in the irradiated control group quickly dropped to the lowest value within 9 days, until death. This showed that the injection of bone marrow cell suspension in the bone-like organ (stem cell reactor) in the irradiated group promoted hematopoietic differentiation in the body, restored hematopoietic function, and promoted the number of red blood cells to be roughly the same as the normal group. The change trends of the number of red blood cells in FIGS. 29 and 30 (7.0 Gy and 8.0 Gy of cobalt 60 irradiation) were almost the same as the weight change trend in FIG. 1.

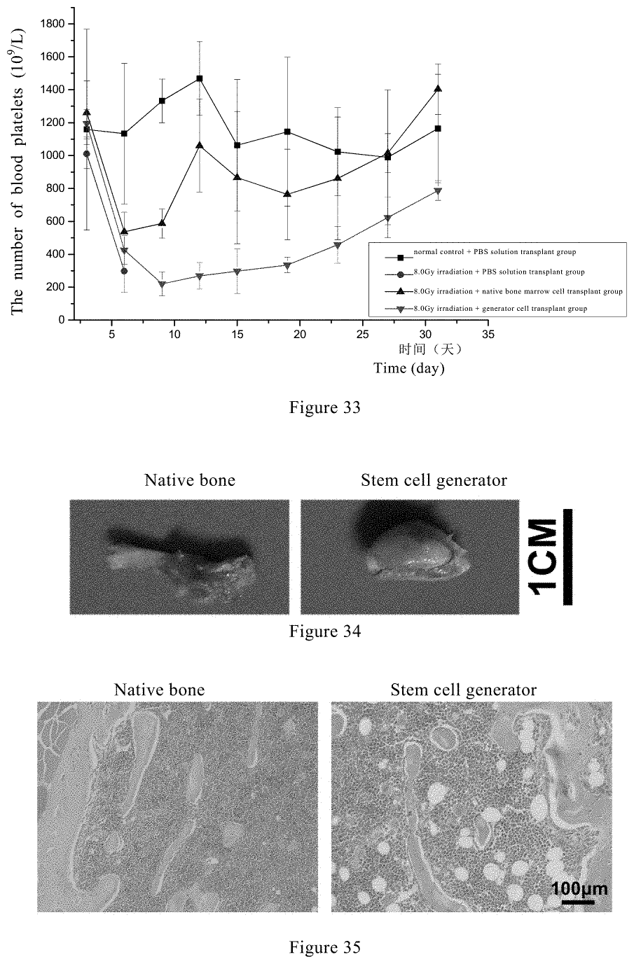

FIGS. 31-33 showed the changes in the number of platelets in the mouse model received different doses of irradiation after treatment. It could be seen from FIG. 31 that both the treatment group and the irradiated control group after irradiation decreased sharply over time, and reached the lowest point at 9 days. After that, the irradiated group remained unchanged until death, while the treatment group increased reversely, and gradually increased over time to the level of the normal control group and restored to the normal level. The obvious difference in FIG. 32 and FIG. 33 was that the recovery degree and speed of radiation treatment group treated by bone marrow cells in the stem cell generator were lower than those of the native bone group, however, the overall trend was the same as that of the normal control group. This was consistent with the weight change trend.

It could be seen that the bone marrow cells in the stem cell generator produced by biomaterial loaded with rhBMP-2 had an effective therapeutic effect on hematopoietic injury caused by radiotherapy and chemotherapy and promoted hematopoiesis. The main effect was that bone marrow cells entered the hematopoietic system and improved the hematopoietic microenvironment, and the various progenitor/stem cells contained therein could normally differentiate into various functions cells to rebuild the blood system.

### Example 10 Evaluation of the Content of Hematopoietic Stem Cells Contained in Stem Cell Generator Manufactured In Vivo

Method: 5 μg of recombinant human bone morphogenetic protein-2 (rhBMP-2) synthesized by eukaryotic or prokaryotic expression system and 1×10⁶ mouse mesenchymal stem cells (mMSCs) were added to collagen gel containing tricalcium phosphate (TCP) (20 mg), and then lyophilized to form an active material containing growth factor. The prepared material was implanted under the skin of the back of 8-week-old SPF C57BL/6 male mice. After 8 weeks of feeding, the stem cell generator was taken out. After the muscles attached to the surface were removed from one part of the stem cell generator, the generator was placed in a mortar containing a little PBS buffer, crushed with a pestle, and passed through a cell sieve to obtain a single cell suspension. The resulting single cell suspension could be used for flow cytometry detection. The other part of stem cell generator was used to take macro photos and make H&E sections. The experiments were grouped as follows.

| Group | Native bone | stem cell generator |
|---|---|---|
| Number | 6 | 6 |

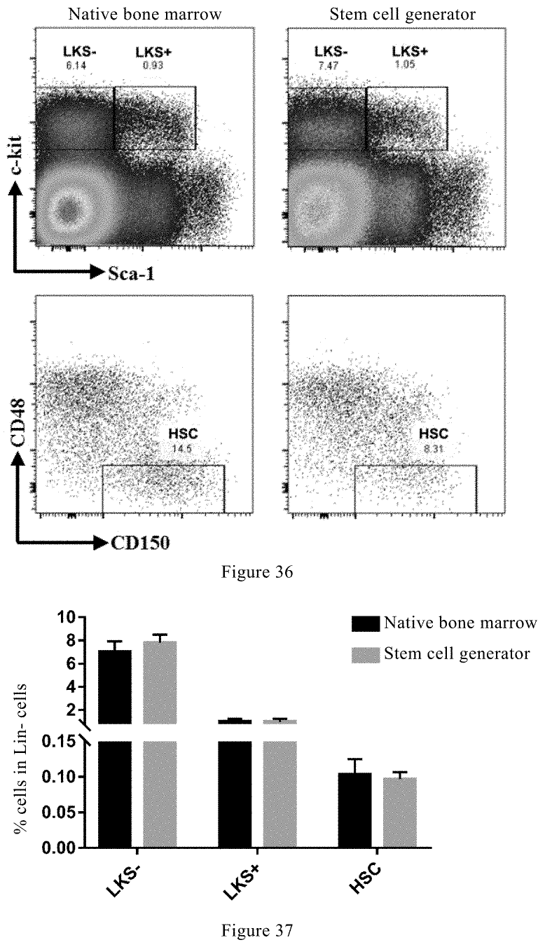

FIG. 34 showed a digital photo of the stem cell generator produced after 8 weeks of subcutaneous implantation on the back. It could be seen that the color of the stem cell generator was similar to that of the native bone, which implied that it contained a large number of red blood cells and had a bone-like morphology. H&E section of the stem cell generator in FIG. 35 further confirmed that the microstructure of the stem cell generator was similar to that of native bone, the structures of cancellous bone and cortical bone were the same, and the bone marrow cavity was filled with bone marrow cells and blood vessels.

FIGS. 36 and 37 showed the flow cytometry correlation analysis of stem cell generator. It could be seen that the stem cell generator and the native bone had similar cell composition, and there was no significant difference between the proportion of LKS− cells, LSK+ cells and hematopoietic stem cells (HSCs) contained in the stem cell generator and the proportion of corresponding cells in the native bone marrow.

The examples illustrated that the constructed stem cell generator had a structure and function similar to native bone marrow, and the hematopoietic stem/progenitor cells contained therein had the potential to treat abnormal hematopoietic function.

All documents mentioned in the present invention are cited as references in this application, as if each document is individually cited as a reference. In addition, it should be understood that after reading the above teaching content of the present invention, those skilled in the art can make various changes or modifications to the present invention, and these equivalent forms also fall within the scope defined by the appended claims of the present application.

1. A stem cell generator, wherein the stem cell generator is formed by implanting a biomaterial loaded with an active substance and/or cell, or a biomaterial with osteoinductive ability into an animal or human body to develop and generate an organoid.

2. A method for constructing the stem cell generator of claim 1, comprising the following steps:
(1) implanting a biomaterial into an animal or human body;
(2) generating an organoid after development in the body to form the stem cell generator, wherein,
the biomaterial is a biomaterial loaded with an active substance and/or cell, or a biomaterial having osteoinductive ability.

3. A method for enriching stem cells, comprising the following steps:
(1) implanting a biomaterial into an animal or human body;
(2) generating an organoid after development in the body and enriching stem cells, wherein
the biomaterial is a biomaterial loaded with an active substance and/or cell, or a biomaterial having osteoinductive ability.

4. The stem cell generator of claim 1, wherein the active substance is bone morphogenetic protein-2 (BMP-2), bone morphogenetic protein-7 (BMP-7), osteogenic peptide, other growth factor or polypeptide having ability to induce bone regeneration and angiogenesis, such as VEGF, PDG, or a combination of the growth factor/polypeptide.

5. The stem cell generator of claim 1, wherein the cell is mesenchymal stem cell, and the mesenchymal stem cell is bone marrow-derived mesenchymal stem cell, adipose-derived mesenchymal stem cell, or mesenchymal stem cell from other sources; other type of cell having osteogenic differentiation ability; a cell assisting mesenchymal stem cell in osteogenic differentiation, such as vascular endothelial cell and the like.

6. The stem cell generator of claim 1, wherein the biomaterial is selected from one of collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix, or a copolymer/blend composition thereof.

7. The stem cell generator of claim 1, wherein the biomaterial is autologous bone or allogeneic bone.

8. The stem cell generator of claim 1, wherein the animal or human body refers to the muscle pocket, muscle space, intra-muscle, subcutis, or dorsal muscle of the abdominal cavity of the animal or human.

9. The stem cell generator of claim 1, wherein the organoid contains stem cell, and the stem cell is hematopoietic stem/progenitor cell, mesenchymal stem cell, endothelial progenitor cell or other type of pluripotent stem cell.

10. Use of the stem cell generator of claim 1 for the manufacture of a material for the prevention and/or treatment of graft-versus-host disease, or hematopoietic injury or for bone marrow transplantation.

11. The method of claim 2, wherein the active substance is bone morphogenetic protein-2 (BMP-2), bone morphogenetic protein-7 (BMP-7), osteogenic peptide, other growth factor or polypeptide having ability to induce bone regeneration and angiogenesis, such as VEGF, PDG, or a combination of the growth factor/polypeptide;
the cell is mesenchymal stem cell, and the mesenchymal stem cell is bone marrow-derived mesenchymal stem cell, adipose-derived mesenchymal stem cell, or mesenchymal stem cell from other sources; other type of cell having osteogenic differentiation ability; a cell assisting mesenchymal stem cell in osteogenic differentiation, such as vascular endothelial cell and the like.

12. The method of claim 2, wherein the biomaterial is selected from one of collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix, or a copolymer/blend composition thereof, or the biomaterial is autologous bone or allogeneic bone.

13. The method of claim 2, the animal or human body refers to the muscle pocket, muscle space, intra-muscle, subcutis, or dorsal muscle of the abdominal cavity of the animal or human.

14. The method of claim 2, wherein the organoid contains stem cell, and the stem cell is hematopoietic stem/progenitor cell, mesenchymal stem cell, endothelial progenitor cell or other type of pluripotent stem cell.

15. The method of claim 3, wherein the active substance is bone morphogenetic protein-2 (BMP-2), bone morphogenetic protein-7 (BMP-7), osteogenic peptide, other growth factor or polypeptide having ability to induce bone regeneration and angiogenesis, such as VEGF, PDG, or a combination of the growth factor/polypeptide;
the cell is mesenchymal stem cell, and the mesenchymal stem cell is bone marrow-derived mesenchymal stem cell, adipose-derived mesenchymal stem cell, or mesenchymal stem cell from other sources; other type of cell having osteogenic differentiation ability; a cell assisting mesenchymal stem cell in osteogenic differentiation, such as vascular endothelial cell and the like.

16. The method of claim 3, wherein the biomaterial is selected from one of collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix, or a copolymer/blend composition thereof, or the biomaterial is autologous bone or allogeneic bone.

17. The method of claim 3, the animal or human body refers to the muscle pocket, muscle space, intra-muscle, subcutis, or dorsal muscle of the abdominal cavity of the animal or human.

18. The method of claim 3, wherein the organoid contains stem cell, and the stem cell is hematopoietic stem/progenitor cell, mesenchymal stem cell, endothelial progenitor cell or other type of pluripotent stem cell.
